(19) United States
(12) Patent Application Publication
(10) Pub. No.: US 2012/0177711 A1
(43) Pub. Date: Jul. 12, 2012

(54) HYDROPHILIC POLYURETHANE UREA SOLUTIONS

(75) Inventors: Sebastian Dörr, Dusseldorf (DE); Jürgen Köcher, Langenfeld (DE); Jörg Hofmann, Krefeld (DE)

(73) Assignee: BAYER MATERIALSCIENCE AG, Leverkusen (DE)

(21) Appl. No.: 13/496,667

(22) PCT Filed: Sep. 13, 2010
(86) PCT No.: PCT/EP10/63379

§ 371 Date: Mar. 16, 2012

(30) Foreign Application Priority Data
Sep. 17, 2009 (EP) 09011854.8

(51) Int. Cl. A61K 8/02; G02C 7/02; C08K 5/05; A01C 1/06; C09D 175/08; C09D 175/12

(52) U.S. Cl. 424/401; 524/590; 524/390; 47/57.6; 351/41

(57) ABSTRACT

The invention relates to a coating composition containing at least one polyurethane urea which is terminated with a copolymer unit of polyethylene oxide and poly-C₄-C₁₂-alkylene oxide.

The present invention relates to a coating composition in the form of a polyurethaneurea solution that can be used for producing hydrophilic coatings. Further subject matter of the present invention is a process for preparing such a coating composition, and, the use of the coating composition, more particularly for the coating of medical devices.

The utilization of medical devices, such as of catheters, can be improved greatly through the equipping thereof with hydrophilic surfaces. The insertion and displacement of urinary or blood vessel catheters is made easier by the fact that hydrophilic surfaces in contact with blood or urine adsorb a water film. This reduces the friction between the catheter surface and the vessel walls, so making the catheter easier to insert and move. Direct watering of the devices prior to the intervention can also be carried out, in order to reduce the friction through the formation of a homogeneous water film. The patients concerned have less pain, and the risk of injury to the vessel walls is reduced as a result. Furthermore, when catheters are used, there is always a risk of blood clots forming.

Suitability for the production of such surfaces is possessed in principle by polyurethane coatings which are produced starting from solutions or dispersions of corresponding polyurethanes.

Thus U.S. Pat. No. 5,589,563 describes the use of coatings having surface-modified end groups for polymers which are used in the biomedical sector and which can also be used for the coating of medical devices. The resulting coatings are produced on the basis of solutions or dispersions, and the polymeric coatings comprise different end groups, selected from amines, fluorinated alkanols, polydimethylsiloxanes and amine-terminated polyethylene oxides. These polymers, however, do not have satisfactory properties as a coating for medical devices, more particularly in respect of the required hydrophilicity.

A disadvantage of aqueous dispersions of the kind described in references including U.S. Pat. No. 5,589,563, moreover, is that the size of the dispersed particles makes the coatings relatively rough. Furthermore, the resultant coatings from aqueous dispersions are generally not sufficiently stable. There is therefore a need for hydrophilic coating systems which exhibit an outstanding hydrophilicity and at the same time have a relatively smooth surface and a high stability.

Polyurethane solutions per se are known from the prior art, but have not—with the exception of the aforementioned polyurethane solutions of U.S. Pat. No. 5,589,563—been used to coat medical devices. As an example, DE 22 21 798 A describes a process for preparing stable and photostable solutions of polyurethaneureas from prepolymers having terminal isocyanate groups and diamines in solvents of low polarity, by reacting prepolymers formed from
a) substantially linear polyhydroxyl compounds having molecular weights of about 500 to 5000,
b) optionally low molecular weight dihydroxy compounds, and
c) aliphatic and/or cycloaliphatic diisocyanates, the molar ratio of hydroxyl groups to isocyanate groups being between about 1:1.5 and 1:5,

in a solvent (mixture) comprising optionally chlorinated aromatic and/or chlorinated aliphatic hydrocarbons and primary, secondary and/or tertiary aliphatic and/or cycloaliphatic alcohols with diamines as chain extenders, where at least 80 mol % of the chain extender is 1,4-diaminocyclohexane having a cis/trans isomer ratio of between 10/90 and 60/40. These polyurethaneurea solutions are used for producing lightfast films and coatings.

DE 22 52 280 A, furthermore, describes a method for the coating of textile substrates by the inversion process, with tie coats and topcoats comprising solutions of aliphatic, segmented polyurethane elastomers which are polycarbonate-containing.

EP 0 125 466 A, furthermore, describes a method for the multi-coat inversion coating of textile substrates, preferably in web form, for producing synthetic leather, comprising at least one topcoat solution and at least one tie coat solution on the basis of polyurethanes.

None of these publications describes a hydrophilic polyurethane resin solution which is used for purposes of coating medical devices and which meets the requirements defined above.

It is an object of the present invention, therefore, to provide a composition which is suitable for coating medical devices with hydrophilic surfaces. Since these surfaces are frequently used in blood contact, the surfaces of these materials ought also to possess good blood compatibility and ought more particularly to reduce the risk of blood clots being formed. Furthermore, the resulting coatings should be smooth and have a good stability.

This invention provides coating compositions in the form of specific polyurethaneurea solutions.

The polyurethaneurea solutions of the invention comprise at least one polyurethaneurea which is terminated with a copolymer unit comprising polyethylene oxide and poly-C₄-C₁₂-alkylene oxide.

In accordance with the invention it has been found that compositions comprising these special polyurethaneureas in solutions are outstandingly suitable for producing coatings on medical devices, to which they give an outstanding hydrophilic coating, form smooth surfaces, have a good stability and at the same time reduce the risk of blood clots forming during treatment with the medical device.

Polyurethaneureas for the purposes of the present invention are polymeric compounds which have
(a) repeat units containing at least two urethane groups, of the following general structure

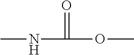

and
(b) at least one repeat unit containing urea groups

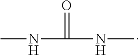

The coating compositions in the form of a solution for use in accordance with the invention are based on polyurethaneureas which have substantially no ionic modification. By this is meant, in the context of the present invention, that the polyurethaneureas for use in accordance with the invention have essentially no ionic groups, such as, more particularly, no sulphonate, carboxylate, phosphate and phosphonate groups.

The term “essentially no ionic groups” means, in the context of the present invention, that the resulting coating of the polyurethaneurea has ionic groups with a fraction of generally at most 2.50% by weight, more particularly at most 2.00% by weight, preferably at most 1.50% by weight, more preferably at most 1.00% by weight, especially at most 0.50% by weight, more especially no ionic groups. It is particularly preferred that the polyurethaneurea has no ionic groups, since high concentrations of ions in organic solution mean that the polymer is no longer sufficiently soluble and hence that stable solutions cannot be obtained. If the polyurethaneurea used in accordance with the invention contains ionic groups, the species in question are preferably carboxylates.

The coating composition of the invention in the form of a solution comprises polyurethaneureas which are preferably substantially linear molecules, but may also be branched, although this is less preferred. In the context of the present invention, by substantially linear molecules are meant systems with a low level of incipient crosslinking, comprising a polyol component having an average hydroxyl functionality of preferably 1.7 to 2.3, more particularly 1.8 to 2.2, more preferably 1.9 to 2.1.

The number-average molecular weight of the polyurethaneureas used with preference in accordance with the invention is preferably 1000 to 200 000, more preferably from 5000 to 100 000. The number-average molecular weight here is measured against polystyrene as standard in dimethylactamide at 30° C.

Polyurethaneureas

The polyurethaneurea-based coating systems for use in accordance with the invention are described in more detail below.

The polyurethaneurea-containing coating compositions of the invention in the form of a solution are prepared by reaction of synthesis components which encompass at least one polycarbonate polyol component, at least one polyisocyanate component, at least one polyoxyalkylene ether component, at least one diamine and/or amino alcohol component and, if desired, a further polyol component.

The individual synthesis components are now described in more detail below.

(a) Polycarbonate Polyol

The polyurethaneurea-based coating composition of the invention in the form of a solution has units which originate from at least one hydroxyl-containing polycarbonate.

Suitable in principle for the introduction of units based on a hydroxyl-containing polycarbonate are polyhydroxyl compounds having an average hydroxyl functionality of 1.7 to 2.3, preferably of 1.8 to 2.2, more preferably of 1.9 to 2.1.

Suitable hydroxyl-containing polycarbonates are polycarbonates of the molecular weight determined by OH number of preferably 400 to 6000 g/mol, more preferably 500 to 5000 g/mol, more particularly of 600 to 3000 g/mol, which are obtainable, for example, through reaction of carbonic acid derivatives, such as diphenyl carbonate, dimethyl carbonate or phosgene, with polyols, preferably diols. Examples of suitable such diols include ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The diol component preferably contains 40% to 100% by weight of hexanediol, preferably 1,6-hexanediol and/or hexanediol derivatives, preferably those which as well as terminal OH groups contain ether or ester groups, examples being products obtained by reaction of 1 mol of hexanediol with at least 1 mol, preferably 1 to 2 mol, of caprolactone or through etherification of hexanediol with itself to give the di- or trihexylene glycol. Polyether-polycarbonate diols as well can be used. The hydroxyl polycarbonates ought to be substantially linear. If desired, however, they may be slightly branched as a result of the incorporation of polyfunctional components, more particularly low molecular weight polyols. Examples of those suitable for this purpose include glycerol, trimethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonates are those based on hexane-1,6-diol, and also on co-diols with a modifying action such as butane-1,4-diol, for example, or else on ε-caprolactone. Further preferred polycarbonate diols are those based on mixtures of hexane-1,6-diol and butane-1,4-diol.

The polycarbonate is preferably of substantially linear construction and has only a slight three-dimensional crosslinking, and so polyurethaneureas are formed which have the abovementioned specification.

(b) Polyisocyanate

The polyurethaneurea-based coating composition of the invention has units which originate from at least one polyisocyanate in the form of a synthesis component.

As polyisocyanates (b) it is possible to use all of the aromatic, araliphatic, aliphatic and cycloaliphatic isocyanates that are known to the skilled person and have an average NCO functionality ≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, which have a carbon backbone (without the NCO groups present) of 3 to 30, preferably 4 to 20, carbon atoms.

Particularly preferred compounds of component (b) conform to the type specified above having aliphatically and/or cycloaliphatically attached NCO groups, such as, for example, bis(isocyanatoalkyl)ethers, bis- and tris(isocyanatoalkyl)benzenes, -toluenes, and -xylenes, propane diisoscyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally as a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component (b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate (IMCl) and/or 4,4′-bis(isocyanatocyclohexyl)methane (H₁₂MDI) or mixtures of these isocyanates. Further examples are derivatives of the above diisocyanates with a uretdione, isocyanurate, urethane, allophanate, biuret, iminooxadiazinedione and/or oxadiazinetrione structure and with more than two NCO groups.

The amount of constituent (b) in the coating composition for use in accordance with the invention is preferably 1.0 to 3.5 mol, more preferably 1,0 to 3.3 mol, more particularly 1.0 to 3.0 mol, based in each case on the constituent (a) of the coating composition for use in accordance with the invention.

(c) Polyoxyalkylene ethers

The polyurethaneurea used in the present invention has units which originate from a copolymer comprising polyethylene oxide and poly-C₄-C₁₂-alkylene oxide in the form of a synthesis component. These copolymer units are present in the form of end groups in the poyurethaneurea and result in hydrophilicizing of the coating composition of the invention.

Nonionically hydrophilicizing compounds (c) are, for example, monofunctional polyalkylene oxide polyether alcohols containing an average 5 to 70, preferably 7 to 55, ethylene oxide units per molecule, of the kind available in conventional manner through alkoxylation of suitable starter molecules (e.g. in Ullmanns Enzyklopädie der technischen Chemie, 4th Edition, Volume 19, Verlag Chemie, Weinheim pp. 31-38).

Examples of suitable starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines such as morpholine, pyrrolidine, piperidine or 1H-pyrazole. Preferred starter molecules are saturated monoalcohols. Particular preference is given to using diethylene glycol monobutyl ether as a starter molecule.

The alkylene oxides, ethylene oxide and C₄-C₁₂-alkylene oxide, can be used in any order or else in a mixture in the alkoxylation reaction.

Polyalkylene oxide polyether alcohols are mixed polyalkylene oxide polyethers of ethylene oxide and C₄-C₁₂-alkylene oxide, whose alkylene oxide units are composed preferably to an extent of at least 30 mol %, more preferably at least 40 mol %, of ethylene oxide units. Preferred non-ionic compounds are monofunctional mixed polyalkylene oxide polyethers which contain at least 40 mol % of ethylene oxide units and not more than 60 mol % of C₄-C₁₂-alkylene oxide units.

The C₄-C₁₂-alkylene oxide unit describes epoxides (oxiranes) with alkyl substituent or alkyl substituents, it being possible for the number of carbon atoms to be from 4 to 12.

C₄-C₁₂-Alkylene oxide units contemplated are preferably those in which the oxirane unit is incorporated in 1,2-position. Examples are 1,2-epoxybutane (butylene oxide), 1,2-epoxypentane, 1,2-epoxyhexane, 1,2-epoxyoctane, 1,2-epoxydecane and 1,2-epoxydodecane. Mixtures of these units are also contemplated. Preferred from the stated selection are 1,2-epoxybutane (butylene oxide), 1,2-epoxypentane and 1,2-epoxyhexane. A particularly preferred C₄-C₁₂-alkylene oxide used is 1,2-epoxybutane (butylene oxide), and with very particular preference the C₄-C₁₂-alkylene oxide used is exclusively 1,2-epoxybutane.

The average molar weight of the polyoxyalkylene ether is preferably 500 g/mol to 5000 g/mol, more preferably 1000 g/mol to 4000 g/mol, more preferably 1000 to 3000 g/mol.

The amount of constituent (c) in the coating composition for use in accordance with the invention is preferably 0.01 to 0.5 mol, more preferably 0.02 to 0.4 mol, more particularly 0.04 to 0.3 mol, based in each case on constituent (a) of the coating composition for use in accordance with the invention.

In accordance with the invention it has been possible to show that the polyurethaneureas with end groups based on mixed polyoxyalkylene ethers comprising polyethylene oxide and poly-C₄-C₁₂-alkylene oxide are especially suitable for producing coatings having a high hydrophilicity. As will be shown later on below, in comparison to polyurethaneureas terminated only by polyethylene oxide, the coatings of the invention effect a significantly low contact angle and are therefore more hydrophilic in form.

(d) Diamine or Amino Alcohol

The polyurethaneurea solution of the invention includes units which originate from at least one diamine or amino alcohol in the form of a synthesis component and serve as what are known as chain extenders.

Examples of such chain extenders are diamines or polyamines and also hydrazides, e.g. hydrazine, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylene-diamine, α,α,α′,α″-tetramethyl-1,3- and -1,4-xylylenediamine and 4,4′-diaminodicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄) di- and tetraalkyldicyclohexylmethanes, e.g. 4,4′-diamino-3,5-diethyl-3′,5′-diisopropyldicyclohexylmethane.

Suitable diamines or amino alcohols are generally low molecular weight diamines or amino alcohols which contain active hydrogen with differing reactivity towards NCO groups, such as compounds which as well as a primary amino group also contain secondary amino groups or which as well as amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, with particular preference, diethanolamine.

The constituent (d) of the coating composition for use in accordance with the invention can be used, in the context of the preparation of the composition, as a chain extender.

The amount of constituent (d) in the coating composition solution of the invention is preferably 0.1 to 1.5 mol, more preferably 0.2 to 1.3 mol, more particularly 0.3 to 1.2 mol, based in each case on constituent (a) of the coating composition for use in accordance with the invention.

(e) Polyols

In a further embodiment the coating composition of the invention in the form of a solution comprises additional units which originate from at least one further polyol in the form of a synthesis component.

The further low molecular weight polyols (e) used to synthesize the polyurethaneureas have the effect, generally, of stiffening and/or branching the polymer chain. The molecular weight is preferably 62 to 500 g/mol, more preferably 62 to 400 g/mol, more particularly 62 to 200 g/mol.

Suitable polyols may contain aliphatic, alicyclic or aromatic groups. Mention may be made here, for example, of the low molecular weight polyols having up to about 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrated bisphenol A (2,2-bis(4-hydroxycyclohexyl)propane), and also trimethylolpropane, glycerol or pentaerythritol, and mixtures of these and, if desired, other low molecular weight polyols as well. Use may also be made of ester diols such as, for example, α-hydroxybutyl-ε-hydroxycaproic acid ester, ω-hydroxyhexyl-γ-hydroxybutyric acid ester, adipic acid (β-hydroxyethyl)ester or terephthalic acid bis(β-hydroxyethyl)ester.

The amount of constituent (e) in the coating composition for use in accordance with the invention is preferably 0.05 to 1.0 mol, more preferably 0.05 to 0.5 mol, more particularly 0.1 to 0.5 mol, based in each case on constituent (a) of the coating composition for use in accordance with the invention.

(f) Further Amine- and/or Hydroxy-Containing Units (Synthesis Component)

The reaction of the isocyanate-containing component (b) with the hydroxy- or amine-functional compounds (a), (c), (d) and, if used, (e) takes place typically with a slight NCO excess being observed over the reactive hydroxy or amine compounds. At the endpoint of the reaction through attainment of a target viscosity, there always still remain residues of active isocyanate. These residues must be blocked in order that no reaction takes place with large polymer chains. Such a reaction leads to the three-dimensional crosslinking and gelling of the batch. The processing of such a coating solution is no longer possible. The batches typically contain large amounts of alcohols. Within a number of hours of the batch at rest or with stirring, at room temperature, these alcohols block the remaining isocyanate groups.

If it is the intention, however, to rapidly block the residual isocyanate content still remaining, the polyurethaneurea coating compositions in the form of a solution provided in accordance with the invention may also comprise monomers (f) in the form of synthesis components, which are located in each case at the chain ends and cap them.

These synthesis components derive on the one hand from monofunctional compounds that are reactive with NCO groups, such as monoamines, more particularly mono-secondary amines, or monoalcohols. Mention may be made here, for example, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the units (f) are used essentially in the coating composition of the invention in the form of a solution to destroy the NCO excess, the amount required is dependant essentially on the amount of the NCO excess, and cannot be specified generally.

During the synthesis, it is preferred that none of these units be used. In this case, any still unreacted isocyanate is preferably reacted via the solvent alcohols contained in very large concentrations to give terminal urethanes.

(g) Further Constituents

Furthermore, the polyurethaneurea coating composition solutions provided in accordance with the invention may comprise further constituents and additives typical for the intended purpose. An example of such are active pharmacological substances, medicaments and additives which promote the release of active pharmacological substances (drug-eluting additives).

Active pharmacological substances or medicaments which may be used in the coatings of the invention on the medical devices and consequently may be contained in the solutions of the invention are, for example, thromboresistant agents, antibiotic agents, antitumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts, and combinations thereof.

Specific examples of such active pharmacological substances or medicaments hence include thromboresistant (non-thrombogenic) agents and other agents for suppressing acute thrombosis, stenosis or late restenosis of the arteries, examples being heparin, streptokinase, urokinase, tissue plasminogen activator, anti-thromboxan-B₂ agent; anti-B-thromboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor can likewise be utilized as a medicament in order to suppress subintimal fibromuscular hyperplasia at the arterial stenosis site, or any other cell growth inhibitor can be utilized at the stenosis site.

The active pharmacological substance or the medicament may also be composed of a vasodilator, in order to counteract vasospasm—for example, an antispasm agent such as papaverine. The medicament may be a vasoactive agent per se, such as calcium antagonists, or α- and β-adrenergic agonists or antagonists. In addition the therapeutic agent may be a biological adhesive such as cyanoacrylate in medical grade, or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for the ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include:
(a) heparin, heparin sulphate, hirudin, hyaluroic acid, chondroitin sulphate, dermatan sulphate, keratan sulphate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;
(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkaloids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof;
(d) antiviral agents such as amantadine, rimantadine, rabavirin, idoxuridine, vidarabin, trifluridine, aciclovir, ganciclovir, zidovudine, phosphonoformates, interferons, their homologues, analogues, fragments, derivatives, pharmaceutical salts and mixtures thereof; and
e) antiinflammatory agents such as, for example, ibuprofen, dexamethasone or methylprednisolone.

In one preferred embodiment the coating composition of the invention in the form of a solution comprises a polyurethaneurea which is at least synthesized from
a) at least one polycarbonate polyol;
b) at least one polyisocyanate;
c) at least one monofunctional polyoxyalkylene ether;
d) at least one diamine or amino alcohol.

In a further preferred embodiment of the present invention the coating composition of the invention in the form of a solution comprises a polyurethaneurea which is at least synthesized from
a) at least one polycarbonate polyol;
b) at least one polyisocyanate;
c) at least one monofunctional polyoxyalkylene ether;
d) at least one diamine or amino alcohol; and
e) at least one polyol.

In a further preferred embodiment of the present invention the coating composition of the invention in the form of a solution comprises a polyurethaneurea which is at least synthesized from
a) at least one polycarbonate polyol;
b) at least one polyisocyanate;
c) at least one monofunctional polyoxyalkylene ether;
d) at least one diamine or amino alcohol;
e) at least one polyol;
f) at least one further amine- and/or hydroxy-containing unit.

The coating compositions of the invention in the form of a solution preferably comprise polyurethaneureas which are at least synthesized from
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.5 mol;
c) at least one monofunctional polyoxyalkylene ether or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.1 to 1.5 mol;
e) if desired, one or more short-chain aliphatic polyols having a molar weight between 62 g/mol and 500 g/mol, in an amount per mole of the polycarbonate polyol of 0.05 to 1 mol; and
f) if desired, amine- or OH-containing units which are located on, and cap, the polymer chain ends.

Preference is further given in accordance with the invention to using polyurethaneureas in the coating composition in the form of a solution which are at least synthesized from
a) at least one polycarbonate polyol having an average molar weight between 500 g/mol and 5000 g/mol and a hydroxyl functionality of 1.8 to 2.2, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.3 mol;
c) at least one monofunctional polyoxyalkylene ether or a mixture of such polyethers, having an average molar weight between 1000 g/mol and 4000 g/mol, in an amount per mole of the polycarbonate polyol of 0.02 to 0.4 mol;
d) at least one aliphatic or cycloaliphatic diamine or at least one amino alcohol, as so-called chain extenders, or mixtures of such compounds in an amount per mole of the polycarbonate polyol of 0.2 to 1.3 mol;
e) if desired, one or more short-chain aliphatic polyols having a molar weight between 62 g/mol and 400 g/mol, in an amount per mole of the polycarbonate polyol of 0.05 to 0.5 mol; and
f) if desired, amine- or OH-containing units which are located on, and cap, the polymer chain ends.

Preference is also further given in accordance with the invention to using polyurethaneureas in the coating solution which are at least synthesized from
a) at least one polycarbonate polyol having an average molar weight between 600 g/mol and 3000 g/mol and a hydroxyl functionality of 1.9 to 2.1, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.0 mol;
c) at least one monofunctional polyoxyalkylene ether or a mixture of such polyethers, having an average molar weight between 1000 g/mol and 3000 g/mol, in an amount per mole of the polycarbonate polyol of 0.04 to 0.3 mol, a mixture of polyethylene oxide and poly-C₄-C₁₂-alkylene oxide being more especially preferred; and
d) at least one aliphatic or cycloaliphatic diamine or at least one amino alcohol, as so-called chain extenders, or mixtures of such compounds in an amount per mole of the polycarbonate polyol of 0.3 to 1.2 mol; and
e) if desired, one or more short-chain aliphatic polyols having a molar weight between 62 g/mol and 400 g/mol, in an amount per mole of the polycarbonate polyol of 0.1 to 0.5 mol.

Use of the Inventive Coating Composition in the Form of a Solution

The coating composition of the invention in the form of a solution can be used to form a coating on a medical device.

The term “medical device” is to be understood broadly in the context of the present invention. Suitable, non-limiting examples of medical devices (including instruments) are contact lenses; cannulas; catheters, for example urology catheters such as urinary catheters or ureteral catheters; central o venous catheters; venous catheters or inlet or outlet catheters; dilation balloons; catheters for angioplasty and biopsy; catheters used for introducing a stent, an embolism filter or a vena caval filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires.

The coating solutions of the invention may be used, furthermore, for producing protective coatings, for example for gloves, stents and other implants; external (extracorporeal) blood lines (blood-carrying pipes); membranes, for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

There are many materials suitable as a substrate of the surface to be coated, such as metals, textiles, ceramics or plastics, the use of plastics being preferred for the production of medical devices.

In accordance with the invention it has been found that it is possible to produce medical devices having very hydrophilic and hence lubricous, blood-compatible surfaces by using aqueous, nonionically stabilized polyurethane dispersions of the type described above to coat the medical devices. The coating compositions described above are obtained preferably as organic solution and are applied to the surface of the medical devices.

In this case, special preference is given to coating solutions which are synthesized from a mixture of polycarbonate polyols and a monofunctional poly-C₄-C₁₂-alkylene oxide polyethylene oxide alcohol.

Preparation of the Coating Solutions

In the context of the present invention it is especially preferred for the coatings on the medical devices to be prepared starting from solutions of the coating composition described in more detail above.

In accordance with the invention it has emerged that the resulting coatings on medical devices differ according to whether the above-described coating composition is prepared starting from a dispersion or from a solution.

The coatings of the invention on medical devices have advantages when they are obtained starting from solutions of the above-described coating compositions.

Without wishing to be tied to one theory it is assumed in accordance with the invention that the filming of the polymers is incomplete because of the particulate structure of the polyurethaneureas in aqueous dispersion. Within the films, the particle structures are generally still perceptible, by means of Atomic Force Microscopy (AFM), for example. Polyurethaneureas from solution yield smoother coatings. On account of the intimate interhooking and interlooping of the polyurethaneurea molecules in organic solution, the dried films as well have greater tensile strength and are more resistant with respect to storage in water.

The medical devices of the invention can be coated by a variety of methods with the hydrophilic polyurethaneurea solutions. Suitable coating techniques for this purpose include, for example, knife coating, printing, transfer coating, spraying, spin coating or dipping.

The organic polyurethaneurea solutions can be prepared by any desired methods. The following procedure, however, has proved to be preferable:

For preparing the polyurethaneurea solutions that are used for coating in accordance with the invention, it is preferred to react the polycarbonate polyol, the polyisocyanate, the monofunctional polyether alcohol and optionally the polyol in the melt or in solution with one another until all of the hydroxyl groups have been consumed.

The stoichiometry used in this case between the individual synthesis components involved in the reaction is evident from the aforementioned quantitative ratios.

The reaction takes place at a temperature of preferably between 60 and 110° C., more preferably 75 to 110° C., more particularly 90 to 110° C., with temperatures of around 110° C. being preferred on account of the rate of the reaction. Higher temperatures can likewise be employed, but then in certain cases, and depending on the individual constituents used, the risk exists of decomposition processes and discolorations occurring in the resultant polymer.

In the case of the prepolymer formed from isocyanate and all of the components containing hydroxyl groups, reaction in the melt is preferred, although the risk exists that the viscosities of the fully reacted mixtures may be too high. In these cases it is also advisable to add solvent. However, as far as possible, no more than approximately 50% by weight of solvent should be present, since otherwise the dilution significantly retards the reaction rate.

In the reaction of isocyanate and the hydroxyl-containing components, the reaction can take place in the melt within a period from 1 hour to 24 hours. Minor addition of solvent quantities results in a retardation, but the reaction times remain within the same time periods.

The sequence of the addition and reaction of the individual constituents may deviate from the sequence indicated above. This may be of advantage especially when the mechanical properties of the resultant coatings are to be modified. If, for example, all of the hydroxyl-containing components are reacted simultaneously, a mixture of hard segments and soft segments is produced. If, for example, the low molecular weight polyol is added after the polycarbonate polyol component, defined blocks are obtained, and this may produce different properties in the resultant coatings. The present invention is therefore not restricted to any particular sequence of addition and/or reaction of the individual constituents of the polyurethaneurea coating.

Then further solvent is added and the optionally dissolved chain extender diamine and/or the dissolved chain extending amino alcohol (synthesis component (d)) are added.

The further addition of the solvent takes place preferably in steps, in order not unnecessarily to retard the reaction, which would take place, for example, at the beginning of the reaction in the case of complete addition of the solvent quantity. In the case of a high solvent content, furthermore, the temperature is limited at the start of the reaction to a comparatively low temperature, which is at least co-determined by the nature of the solvent. This as well leads to a retardation of the reaction.

When the target viscosity has been attained, the residues of NCO still remaining can be blocked by means of a monofunctional aliphatic amine. It is preferred to block the remaining isocyanate groups by reaction with the alcohols present in the solvent mixture.

Solvents suitable for preparing and using the polyurethaneurea solutions of the invention include all conceivable solvents and solvent mixtures such as dimethylformamide, N-methylacetamide, tetramethylurea, N-methylpyrrolidone, aromatic solvents such as toluene, linear and cyclic o esters, ethers, ketones and alcohols. Examples of esters and ketones are, for example, ethyl acetate, butyl acetate, acetone, γ-butyrolactone, methyl ethyl ketone and methyl isobutyl ketone.

Preference is given to mixtures of alcohols with toluene. Examples of the alcohols which are used together with the toluene are ethanol, n-propanol, isopropanol and 1-methyoxy-2-propanol.

In the reaction, generally speaking, the amount of solvent used is such as to give solutions with a strength of approximately 10% to 50% by weight, more preferably approximately 15% to 45% by weight, more preferably approximately 20% to 40% by weight.

The solids content of the polyurethaneurea solutions is generally between 5% to 60% by weight, preferably 10% to 40% by weight. For coating experiments the polyurethaneurea solutions can be diluted arbitrarily with toluene/alcohol mixtures, in order to allow the thickness of the coating to be varied. All concentrations from 1% to 60% by weight are possible; preference is given to concentrations in the 1% to 40% by weight range.

In this context it is possible to attain any desired coat thicknesses, such as, for example from a few 100 nm up to several 100 μm, although higher and lower thicknesses are possible in the context of the present invention.

Further additions, such as antioxidants or pigments, for example, may likewise be used. It is also possible if desired, furthermore, to use further additions such as hand assistants, dyes, matting agents, UV stabilizers, light stabilizers, hydrophobicizing agents and/or flow control assistants.

Starting from these solutions, then, medical coatings are produced by the processes described above.

A wide variety of substrates can be coated, such as metals, textiles, ceramics and plastics. Preference is given to coating medical devices manufactured from plastic or from metals. Examples of metals include the following: medical stainless steel and nickel titanium alloys. Many polymer materials are conceivable from which the medical device may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethaneureas to the medical device, further suitable coatings may be applied as a base before these hydrophilic coating materials are applied.

The medical devices can be coated with the hydrophilic polyurethaneurea dispersions by a variety of methods. Suitable coating techniques include knifecoating, printing, transfer coating, spraying, spin coating or dipping.

In addition to the hydrophilic properties of the improvement of slip, the coating compositions provided in accordance with the invention are also distinguished by a high level of blood compatibility. As a result, working with these coatings is also advantageous, particularly in blood contact. In comparison to polymers of the prior art, the materials exhibit reduced coagulation tendency in blood contact.

Besides the stated applications in the medical sector, the systems of the invention can also be used for coating technical substrates in the non-medical sector, for producing easy-to-clean or self-cleaning surfaces, for coating glazing systems and optical glasses and lenses, for coating substrates in the hygiene sector, for coating packaging materials, for reducing the growth on the coated surfaces, for coating above-water and underwater substrates for reducing the substrates’ frictional resistance toward water, for preparing substrates for printing, for preparing formulations for cosmetic applications or for producing active-ingredient-releasing systems for the coating of seeds.

The advantages of the catheters of the invention with the hydrophilic polyurethaneurea coatings are set out by means of comparative experiments in the following examples.

### EXAMPLES

The NCO content of the resins described in the inventive and comparative examples was determined by titration in accordance with DIN EN ISO 11909.

The solids contents were determined in accordance with DIN-EN ISO 3251. 1 g of polyurethaneurea solution was dried at 115° C. to constant weight (15-20 min) using an infrared dryer.

The average particle sizes of the polyurethaneurea solutions are measured using the High Performance Particle Sizer (HPPS 3.3) from Malvern Instruments.

Unless noted otherwise, amounts indicated in % are % by weight and relate to the solutions obtained.

Viscosities were measured using the Physics MCR 51 rheometer from Anton Paar GmbH, Ostfildern, Germany.

Substances and Abbreviations used:

Desmophen C2200: Polycarbonate polyol, OH number 56 mg KOH/g, number-average molecular weight 2000 g/mol (Bayer, MaterialScience AG, Leverkusen, DE)

### Example 1

This example describes the preparation of an inventive polyurethaneurea solution.

Desmophen C 2200 (195.4 g), 30.0 g of monofunctional polyether based on ethylene oxide/butylene oxide (number-average molecular weight 2250 g/mol, OH number 25 mg KOH/g, butylene oxide (=1,2-epoxybutane) fraction: 25% by weight) and 47.8 g of 4,4′-bis(isocyanatocyclohexyl)methane (H₁₂MDI) were reacted at 110° C. to a constant NCO content of 2.4%. The product was left to cool and diluted with 350.0 g of toluene and 200 g of isopropanol. At room temperature, a solution of 10.25 g of isophoronediamine in 94.0 g of 1-methoxypropan-2-ol was added. After the end of the build-up in molar weight and attainment of the desired viscosity range, stirring was continued for 4 hours more in order to block the residual isocyanate content with isopropanol. This gave 930 g of a 31% strength polyurethaneurea solution in toluene/isopropanol/1-methoxypropan-2-ol, having a viscosity of 22 400 mPas at 23° C.

### Example 2

### Production of the Coatings and Measurement of the Static Contact Angle

The coatings for the measurement of the static contact angle were produced on purified glass plates using a knife coater (knife coater gap: 210 micrometers). This gave a homogeneous coating, which was dried at 100° C. for 1 h and then at 50° C. for 24 h or alternatively only at 23° C. The coated glass plates obtained were subjected directly to a contact angle measurement.

A static contact angle measurement was performed on the resulting coatings on the glass plates. Using the video contact angle measuring instrument OCA20 from Dataphysics, with computer-controlled injection, 10 drops of Millipore water were placed on the specimen, and their static wetting angle was measured. Beforehand, using an antistatic dryer, the static charge (if present) on the sample surface was removed.

TABLE 1
Static contact angle measurements

| PU FILM made from | Contact angle [°] |
|---|---|
| Inventive Example 1 (drying 100° C. and 50° C.) | 29.8 |
| Inventive Example 1 (drying 23° C.) | 32.4 |

As Table 1 shows, the polycarbonate-containing coating of Inventive Example 1 gives an extremely hydrophilic coating with a static contact angle ≦40°.

1-15. (canceled)

16. Coating composition in the form of a solution containing at least one polyurethaneurea, characterized in that the polyurethaneurea is terminated with a copolymer unit comprising polyethylene oxide and poly-C₄-C₁₂-alkylene oxide.

17. Coating composition according to claim 16, characterized in that the polyurethaneurea comprises units which originate from at least one hydroxyl-group-containing polycarbonate.

18. Coating composition according to claim 16, characterized in that the polyurethaneurea has units which originate from aliphatic or cycloaliphatic polyisocyanates.

19. Coating composition according to claim 16, characterized in that the polyurethaneurea has units which originate from at least one polyol.

20. Coating composition according to claim 16, characterized in that the polyurethaneurea has units which originate from at least one diamine or amine alcohol.

21. Coating composition according to claim 16, characterized in that the polyurethaneurea has units which originate from further hydroxyl- and/or amine-containing synthesis components.

22. Coating composition according to claim 16, characterized in that the polyurethaneurea is at least synthesized from the following synthesis components
a) at least one polycarbonate polyol;
b) at least one polyisocyanate;
c) at least one monofunctional polyoxyalkylene ether; and
d) at least one diamine or one amino alcohol.

23. Coating composition according to claim 22, characterized in that polyurethaneurea further comprises synthesis components comprising
e) at least one polyol.

24. Coating composition according to claim 16, characterized in that the polyurethaneurea is at least synthesized from the following synthesis components:
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.5 mol;
c) at least one monofunctional polyoxyalkylene ether or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.1 to 1.5 mol; and
e) if desired, one or more short-chain aliphatic polyols having a molar weight between 62 g/mol and 500 g/mol, in an amount per mole of the polycarbonate polyol of 0.5 to 1.0 mol.

25. Process for preparing a polyurethaneurea solution according to claim 16, characterized by the following process steps:
(I) reacting the polycarbonate polyol, the polyisocyanate and the monofunctional polyoxyalkylene ether and optionally the polyol in the melt or in the presence of a solvent in solution until all of the hydroxyl groups are consumed;
(II) adding further solvent and optionally adding the dissolved diamine or the optionally dissolved amino alcohol; and
(III) optionally blocking the residues of NCO groups still left after the target viscosity has been achieved, with a monofunctional aliphatic amine.

26. Process according to claim 25, characterized in that the solvent is selected from the group consisting of N-ethylpyrrolidone, dimethylformamide, N-methylacetamide, tetramethylurea, N-methylpyrrolidone, y-butyrolactone, aromatic solvents, linear and cyclic esters, ethers, ketones, alcohols and mixtures thereof

27. Coating composition in the form of a solution, obtainable according to claim 25.

28. Use of a coating composition according to claim 16 for coating at least one medical device.

29. Use of a coating composition according to claim 16 for coating technical substrates in the non-medical sector, for producing easy-to-clean or self-cleaning surfaces, for coating glazing systems and optical glasses and lenses, for coating substrates in the hygiene sector, for coating packaging materials, for reducing growth on the coated surfaces, for the coating of above-water and underwater substrates in order to reduce the substrates' frictional resistance toward water, for preparing substrates for printing, for producing formulations for cosmetic applications or for producing active-ingredient-releasing systems for the coating of seeds.

30. Coating on a substrate, obtainable by applying a coating composition according to claim 16 to the substrate and drying it.
